(19) United States
(12) Patent Application Publication
(10) Pub. No.: US 2023/0290093 A1
(43) Pub. Date: Sep. 14, 2023

(54) DATA PROCESSING METHOD

(71) Applicant: MEDIT CORP., Seoul (KR)

(72) Inventor: Dong Hoon LEE, Seoul (KR)

(73) Assignee: MEDIT CORP., Seoul (KR)

(21) Appl. No.: 18/035,461

(22) PCT Filed: Nov. 2, 2021
(86) PCT No.: PCT/KR2021/015667

§ 371 Date: May 4, 2023

(30) Foreign Application Priority Data
Nov. 5, 2020 (KR) 10-2020-0146535
Oct. 15, 2021 (KR) 10-2021-0137277

(51) Int. Cl. G06T 19/20; A61C 13/34

(52) U.S. Cl. CPC G06T 19/20; A61C 13/34; G06T 2210/41; G06T 2219/2012

(57) ABSTRACT

A data processing method according to the present disclosure includes: distinguishing, in a 3D model, an analysis region including at least one tooth region; and determining a degree of completeness of the 3D model based on the analysis region.

### CROSS REFERENCE TO RELATED APPLICATIONS

This application is a National Stage of International Application No. PCT/KR2021/015667 filed Nov. 2, 2021, claiming priority based on Korean Patent Application No. 10-2020-0146535 filed Nov. 5, 2020 and Korean Patent Application No. 10-2021-0137277 filed Oct. 15, 2021, the entire disclosures of which are incorporated herein by reference.

### TECHNICAL FIELD

The present disclosure relates to a data processing method, and more particularly, to a data processing method for acquiring a three dimensional (3D) model of an object and determining a degree of completeness of the 3D model.

### BACKGROUND

A 3D scanning technique is used in various industrial fields such as measurement, inspection, reverse engineering, content creation, CAD/CAM for dental treatment, medical devices, and the like. The applicability of the 3D scanning technique is further expanding as a result of improvement in scanning performance due to the development of computing technology. In particular, in the field of dental treatment, the 3D scanning technique is used for treatment of patients. Therefore, a 3D model obtained through 3D scanning is required to have a high degree of completeness.

In a process of creating a 3D model through a 3D scanner, the 3D scanner acquires the entire 3D model data by converting image data (in 2D or 3D) acquired through imaging of a measurement object into a 3D model. In addition, as the measurement object is closely imaged, the number of images acquired by the 3D scanner increases. Accordingly, the reliability of the final data of the 3D model converted in real time is improved.

Conventionally, the degree of completeness and/or reliability of final data for a measurement object has depended on a user’s personal determination. However, since the user’s personal determination is ambiguous in its standard and relies only on senses, it is difficult to trust the degree of completeness of final data.

In order to improve this problem, a method of visually displaying reliability by assigning a predetermined color or applying a pattern to a 3D model has recently been used. For example, there has been a user interface (UI) that displays a low reliability region in red, a medium reliability region in yellow, and a high reliability region in green according to the reliability of data constituting a 3D model.

However, this method has a disadvantage in that the user needs to keep looking at a display device on which the user interface is continuously displayed. As in a user interface screen shown in FIG. 1, a so-called “reliability map” method has been used to indicate the reliability of data in colors on a 3D model. In the reliability map method, in order to check the actual color of the 3D model, the user has to click a button to switch the display mode. For example, referring to FIG. 1, the reliability of the 3D model may be expressed in a first reliability color RD1, a second reliability color RD2, and a third reliability color RD3. The reliability colors RD1, RD2, and RD3 are exemplary. Various means (patterns, etc.) for indicating reliability may be used.

In addition, the user has to click a mode switching button 12 to switch between a mode representing the reliability of the 3D model and a mode representing the actual color of the 3D model to alternatively check the information (reliability or actual color of the 3D model) displayed in each mode. This process requires the user to perform unnecessary operations and prevents the user from quickly analyzing the 3D model.

In addition, even if the mode representing the reliability is used, the user still has no choice but to determine the degree of completeness of the 3D model by the user’s visual determination. This has a problem of not guaranteeing the degree of completeness of the 3D model above a certain level.

Therefore, a method for solving the aforementioned disadvantages is needed.

### SUMMARY

The present disclosure provides a data processing method that does not require a user’s personal determination and that can determine the reliability of quantitative data by a system itself and provide a feedback of a determination result to a user.

The technical problems of the present disclosure are not limited to those mentioned above. Other technical problems not mentioned herein may be clearly understood by those skilled in the art from the description below.

In order to achieve the above-described purpose, a data processing method according to the present disclosure may include: distinguishing, in a 3D model, an analysis region including at least one tooth region; and determining a degree of completeness of the 3D model based on the analysis region.

In addition, the data processing method according to the present disclosure may further include other additional processes in addition to the above-described processes. This enables the user to easily check the degree of completeness of the 3D model.

By using the data processing method according to the present disclosure, the user can easily obtain a 3D model having sufficient reliability.

In addition, by using the data processing method according to the present disclosure, the degree of completeness is determined for an analysis region rather than the entire 3D model. Therefore, it is possible to reduce the data processing time.

In addition, by using the data processing method according to the present disclosure, the user can easily check whether the 3D model has a reliable degree of completeness based on the accurately calculated and determined result without resorting to arbitrary determination.

In addition, by using the data processing method according to the present disclosure, a 3D model can be acquired by precisely scanning a more important tooth by applying different degree of completeness determination thresholds for individual tooth regions.

In addition, by using the data processing method according to the present disclosure, the user can visually and easily check parts of the 3D model detected as attention regions, and the time and effort required when performing an additional scan to minimize the attention regions can be saved.

### BRIEF DESCRIPTION OF DRAWINGS

FIG. 1 is a view for explaining a reliability map according to a conventional technique.

FIG. 2 is a flowchart showing a data processing method according to the present disclosure.

FIG. 3 is a view for explaining a process of setting an analysis region.

FIG. 4 is a view for explaining a process of acquiring a 3D model by scanning an object.

FIG. 5 is a detailed flowchart showing a step of distinguishing an analysis region in the data processing method according to the present disclosure.

FIG. 6 is a view for explaining a distinguishing criterion used to distinguish individual tooth regions of a teeth region.

FIG. 7 is a view for explaining a process of detecting a blank region in an attention region.

FIG. 8 is a view for explaining another process of detecting a blank region in the attention region.

FIG. 9 is a view for explaining a step of generating a feedback based on a result of determining a degree of completeness in the data processing method according to the present disclosure.

FIG. 10 is a view for explaining an attention region fed back to the user.

FIG. 11 is a flowchart showing a data processing method according to another embodiment of the present disclosure.

FIG. 12 is a view for explaining a process of setting an analysis region.

FIG. 13 is a schematic configuration diagram showing a data processing apparatus that performs the data processing method according to the present disclosure.

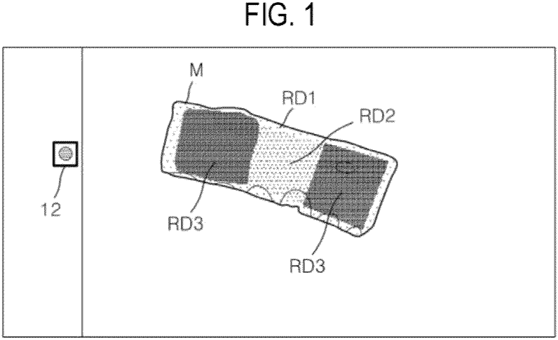

### DESCRIPTION OF REFERENCE NUMERALS

S110: Step of setting an analysis region
S120: Step of acquiring a 3D model
S130: Step of distinguishing the analysis region in the 3D model
S140: Step of determining a degree of completeness of the 3D model based on the analysis region
S150: Generating a feedback based on a result of the determination of the degree of completeness
100: Analysis region 200: 3D model
300: Template 600: User interface screen
700: Feedback means 710: Completeness display means
720: Attention region display means
900: Data processing apparatus

### DETAILED DESCRIPTION

Hereinafter, some embodiments of the present disclosure will be described in detail through exemplary drawings. It should be noted that when giving reference numerals to the components of each drawing, the same components have the same numerals as much as possible even if they are depicted on different drawings. In addition, when describing embodiments of the present disclosure, if it is determined that detailed description of a related known configuration or function hinders understanding of the embodiments of the present disclosure, the detailed description thereof will be omitted.

When describing the components of the embodiments of the present disclosure, terms such as first, second, A, B, (a), and (b) may be used. These terms are used to merely distinguish the corresponding components from other components, and the nature, sequence, or order of the corresponding components is not limited by the terms. In addition, unless defined otherwise, all terms used herein, including technical or scientific terms, have the same meaning as commonly understood by a person of ordinary skill in the art to which the present disclosure belongs. Terms such as those defined in the commonly used dictionaries should be interpreted as having a meaning consistent with the meaning in the context of the related art, and should not be interpreted in an ideal or excessively formal meaning unless explicitly defined in the subject application.

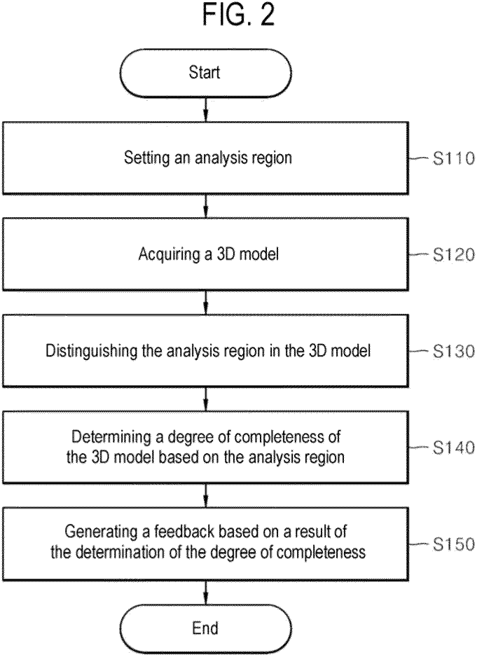

FIG. 2 is a flowchart showing a data processing method according to the present disclosure.

Referring to FIG. 2, the data processing method according to the present disclosure includes a step S110 of setting an analysis region, a step S120 of acquiring a 3D model, a step S130 of distinguishing the analysis region, a step S140 of determining a degree of completeness, and a step S150 of generating a feedback. The data processing method according to the present disclosure may be performed according to the above steps and sub-steps of the above steps.

Hereinafter, each step of the data processing method according to the present disclosure will be described in detail.

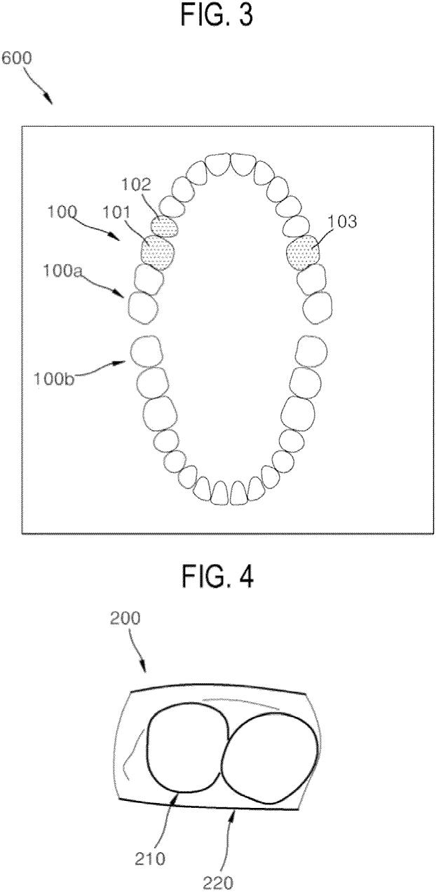

FIG. 3 is a view for explaining a process of setting an analysis region.

Referring to FIGS. 2 and 3, the data processing method according to the present disclosure includes the step S110 of setting an analysis region. In this case, an analysis region 100 corresponds to a region for determining the completeness of a 3D model. In the data processing method according to the present disclosure, the completeness of the 3D model is determined not based on the entire 3D model but based on a part of the 3D model set by the analysis region 100. As shown in FIG. 3, a standard oral cavity shape for setting the analysis region 100 is displayed on a user interface screen 600. The standard oral cavity shape is different from the 3D model representing the object, and may be a 2D shape or a 3D shape schematically showing a general oral arrangement for setting the analysis region 100 described later. As shown in FIG. 3, portions to be set as the analysis region 100 may be selected from the standard oral cavity shape displayed on the user interface screen 600. For example, the analysis region 100 may include an upper jaw analysis region 100a and a lower jaw analysis region 100b. The user may select at least one of the teeth disposed in the upper jaw in the upper jaw analysis region 100a as the analysis region 100, or may select at least one of the teeth disposed in the lower jaw in the lower jaw analysis region 100b as the analysis region 100. That is, the analysis region 100 may be set to at least a portion of the upper jaw analysis region 100a and/or at least a portion of the lower jaw analysis region 100b. For example, as shown in FIG. 3, the analysis region 100 may be determined as a first tooth 101, a second tooth 102 and a third tooth 103 in the upper jaw analysis region 100a.

FIG. 4 is a view for explaining a process of acquiring a 3D model by scanning an object.

Referring to FIGS. 2 and 4, the data processing method according to the present disclosure may include the step S120 of acquiring a 3D model of an object by scanning the object. The object may represent a tooth of a patient. For example, the object may be a patient’s actual oral cavity or a plaster model created by applying plaster to a mold obtained by taking an impression of the patient’s oral cavity.

The user may scan an object using a scanner and obtain a 3D model 200 of the object. In this case, the scanner may be a 3D scanner. For example, the 3D scanner may be a table scanner that obtains the 3D model 200 of the object through a camera disposed on one side by arranging the object on a tray and rotating or tilting the tray, or may be a handheld scanner that obtains the 3D model 200 by directly holding the object by a user and scanning the object at various angles and distances.

Since the 3D model 200 obtained using the scanner represents the patient’s oral cavity or the plaster model molding the patient’s oral cavity, the 3D model 200 may include a tooth region 210 representing a tooth and a gingival region 220 representing a gingiva. The tooth region 210 and the gingival region 220 may be distinguished on a region-by-region basis through a predetermined distinguishing criterion. The distinguishing process will be described later.

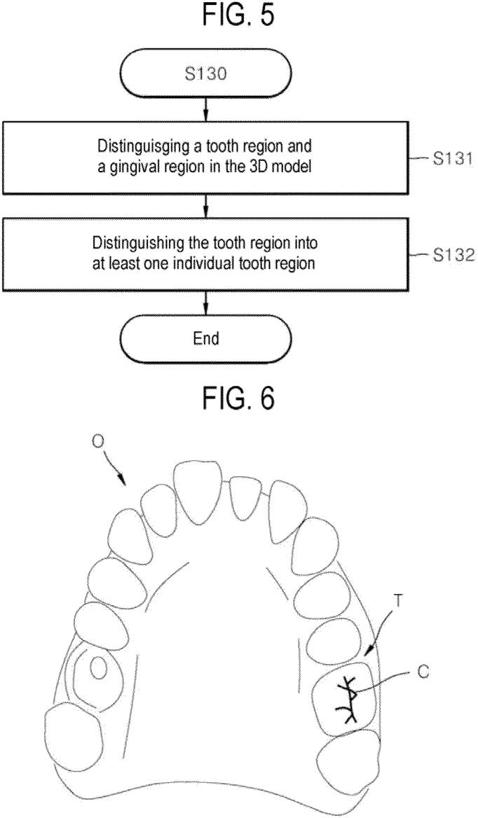

FIG. 5 is a detailed flowchart showing a step of distinguishing an analysis region in the data processing method according to the present disclosure. FIG. 6 is a view for explaining a distinguishing criterion used to distinguish individual tooth regions of a teeth region.

Referring to FIGS. 2 to 5, the data processing method according to the present disclosure may include the step S130 of distinguishing an analysis region including at least one tooth region 210 in the acquired 3D model 200. The step S130 of distinguishing the analysis region may mean determining at least some regions of the 3D model 200 used for determining the degree of completeness from the entire 3D model 200. For example, when all of the teeth of the object are set as the analysis region 100 in the step S110 of setting the analysis region described above, the tooth region 210 of the 3D model 200 may be distinguished and determined as the analysis region 100. For example, the tooth region 210 may include the entire tooth region of the 3D model 200. That is, the tooth region 210 may refer to all regions except the gingival region 220 of the 3D model 200. For example, when all the teeth are set as the analysis region 100 in the portion representing the upper jaw and the portion representing the lower jaw in the 3D model 200, the tooth region 210 representing all the teeth of the upper jaw in the 3D model 200 and the tooth region 210 representing all the teeth of the lower jaw in the 3D model 200 may be determined as the analysis region 100.

If necessary, when the analysis region 200 is set as the entire patient’s teeth, the data processing method according to the present disclosure may determine the distinguished tooth region 210 and at least a portion of the gingival region 220 surrounding the tooth region 210 as the analysis region 200. For example, the analysis region 200 may be determined to include a portion of the gingival region 220 around the tooth region 210 within a predetermined distance from the tooth region 210. By determining the analysis region 200 to include a portion of the gingival region 220, it is possible to determine a degree of completeness of the 3D model including not only a portion representing the tooth itself but also a portion of the gingiva where the tooth is present. In this way, the user can easily check whether a margin line of a tooth required due to tooth preparation or the like is precisely scanned.

Hereinafter, sub-steps of the step S130 of distinguishing the analysis region will be described in detail.

In the step S130 of distinguishing the analysis region, the 3D model 200 may be distinguished into a tooth region 210 representing a tooth and a gingival region 220 representing a gingiva. That is, the step S130 of distinguishing the analysis region may include a step S131 of distinguishing a tooth region and a gingival region in a 3D model. In order to distinguish the 3D model 200 into the tooth region 210 and the gingival region 220, at least one among predetermined distinguishing information may be used. The distinguishing information may include at least one among color information and curvature information. For example, when the object is the patient’s actual oral cavity, the gingiva may have a bright red or pink color, and the tooth may have a white or ivory color. The 3D model 200 may be displayed as a plurality of voxels. Each voxel may include color information on a position of the 3D model 200 corresponding to the object. When color information is used as the distinguishing information, the 3D model 200 may be distinguished into a region corresponding to the tooth region 210 or a region corresponding to the gingival region 220 according to color information of each voxel. However, the present disclosure is not limited thereto. The tooth region 210 and the gingival region 220 of the 3D model 200 may be distinguished based on the 2D image data (not shown) of a plane acquired to create the 3D model 200 of the object in the step S120 of acquiring the 3D model. For example, the 2D image data may include a plurality of pixels, and each pixel may include color information on a corresponding position of the object. According to the color information of each pixel, the color information may be allocated to voxels generated by converting the 2D image data into a 3D model. The 3D model 200 may be distinguished into the tooth region 210 and the gingival region 220.

In addition, when curvature information is used as distinguishing information, a curvature value at the boundary between the tooth region 210 and the gingival region 220 of the 3D model 200 is larger than a curvature value in other portions of the 3D model 200. Accordingly, the 3D model 200 may be distinguished into the tooth region 210 and the gingival region 220 by using a portion having a curvature value equal to or larger than a predetermined threshold value as a boundary.

If necessary, when noise data such as saliva and soft tissue is included in the 3D model 200 acquired by scanning the object, the step S130 of distinguishing the analysis region (S130) may additionally distinguish a noise region (not shown) in addition to the tooth region 210 and the gingival region 220. The noise region may be excluded from the determination criterion of the 3D model 200. Thus, the noise region is not determined as the analysis region 100. In addition, the noise regions may be individually or separately selected and deleted from the 3D model 200. Accordingly, it is possible to improve the overall degree of completeness of the 3D model 200.

Referring to FIGS. 5 and 6, the step S130 of distinguishing the analysis region may further include a step S132 of distinguishing a tooth region into at least one individual tooth region. The step S132 of distinguishing the tooth region into at least one individual tooth region may be performed only when the entire tooth region is not set but only some teeth are selected in the step S110 of setting the analysis region.

As shown in FIG. 6, the object O may include at least one tooth T. Each tooth T may have unique surface curvature C information. For example, the first molar, the second molar, the first premolar, the second premolar, the canine, and the anterior teeth have different pieces of surface curvature information, which may be pre-stored in a database part of a data processing system in which the data processing method according to the present disclosure is performed. In addition, the surface curvature information may be pre-stored in the database part so that a dental formula corresponding to each curvature information can be detected by using a deep learning technique. Accordingly, the tooth region 210 may be distinguished into individual tooth regions representing individual teeth according to a dental formula distinguishing criterion including surface curvature C information of the tooth T.

In addition, tooth size information, tooth shape information, and the like may be used together as a dental formula distinguishing criterion for distinguishing the tooth region 210 into individual tooth regions. For example, the first molar may be larger than the first premolar, and the first premolar may be larger than the canine. According to the size relationship between the teeth, the dental formula of the tooth constituting the 3D model 200 may be distinguished, and the individual tooth regions may be determined and used as a basis for determining the degree of completeness. Meanwhile, one of a FDI method, a Palmer method and a universal numbering system method may be used as a method of assigning a dental formula. Other dental formula assigning methods not listed may also be used. The type of dental formula assigning method is not limited.

Meanwhile, in order to distinguish the tooth region 210 into individual tooth regions, a template pre-stored in the database part may be used. The template is data representing standard teeth stored in the database part. The template has information about a dental formula. Accordingly, by matching the 3D model 200 with a template having a similar shape, the teeth formed in the tooth region 210 may be distinguished into individual tooth regions.

Alternatively, in order to distinguish the tooth region 210 into individual tooth regions, a curvature value appearing in the contour (or boundary) of each tooth may be used. For example, a large curvature value appears in the contour of an individual tooth. Therefore, individual tooth regions may be distinguished for each dental formula through the portions of the 3D model 200 having curvature values equal to or larger than a predetermined threshold value.

As described above, the step S132 of distinguishing individual tooth regions may be performed after the tooth region 210 and the gingival region 220 are distinguished in the 3D model 200. However, the present disclosure is not necessarily limited thereto. That is, the step S132 of distinguishing individual tooth regions may be performed directly by omitting the step S131 in which the 3D model 200 is distinguished into the tooth region 210 and the gingival region 220.

When the analysis region 100 is distinguished in the 3D model 200, the step S140 of determining the degree of completeness of the 3D model 200 based on the analysis region 100 may be performed. For example, the degree of completeness of the 3D model 200 may indicate a degree to which data is accumulated to the extent that the 3D model 200 can be trusted.

Data reliability for determining the degree of completeness of the 3D model 200 will be described. A plurality of scan data is required in order to create the 3D model 200. For example, the scan data may be shots of 3D data. At least some of the scan data may overlap each other, and the overlapping portion of the scan data may represent the same portion of the object. Accordingly, the scan data may be aligned by overlapping portions, and the entire aligned scan data may become the 3D model 200.

Meanwhile, when the scan data is aligned, a portion where a relatively large number of data overlaps may have high reliability, and a portion where a relatively small number of data overlaps may have low reliability. In some cases, when the 3D model 200 is created, there may be a portion where data is missing. The missing portion remains blank.

At this time, the region remaining blank and the region having low reliability in the 3D model 200 may be referred to as an “attention region.” More specifically, the attention region may include a blank region in the 3D model 200 for which scan data is not input and a low-density region for which scan data is input as being below a predetermined threshold density in the 3D model 200.

Hereinafter, the blank region of the attention region will be described in more detail.

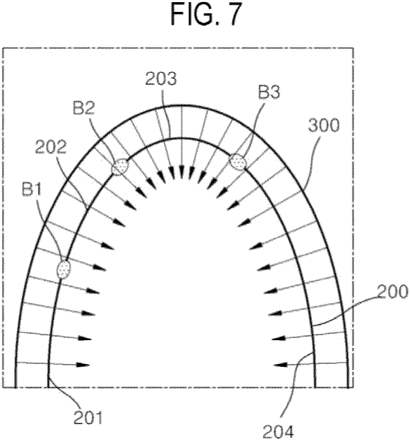

FIG. 7 shows simplified views of the 3D model 200 and a template 300 for explaining a process of detecting a blank region B in the attention region.

Referring to FIG. 7, the blank region B may be detected by using the 3D model 200 and the template 300. For example, the 3D model 200 may be aligned with the template 300 stored in the database part. The template 300 represents a standard shape of the object O, and the template 300 may theoretically be a model in which the attention region does not exist. Meanwhile, the 3D model 200 obtained by scanning the object O is aligned with the template 300, and at least one light beam is generated from the surface of the template 300. For example, a plurality of light beams may be generated from the surface of the template 300. More specifically, the light beams may be generated from vertices of all meshes constituting the surface of the template 300. The generated light beams travel in a direction normal to the surface of the template 300, and at least some of the light beams may meet the surface of the 3D model 200. The light beams may travel in two directions from the surface of the template 300 or in one direction from the template 300 toward the 3D model 200. At this time, there may be portions through which the light beams pass without meeting the surface of the 3D model 200, and these portions correspond to the portions to which data is not input (see the hatched portions in FIG. 7). Accordingly, the portions through which the light beams pass without meeting the surface of the 3D model 200 may be defined as the blank region B. The process of generating light beams to detect the blank regions B and checking whether the light beams intersect the 3D model and the template in this way is called an intersection test.

For example, as shown in FIG. 7, a first blank region B1 may be detected between the first 3D model surface 201 and the second 3D model surface 202, a second blank region B2 may be detected between the second 3D model surface 202 and the third 3D model surface 203, and a third blank region B3 may be detected between the third 3D model surface 203 and the fourth 3D model surface 204. Meanwhile, the number of light beams generated from the surface of the template 300 may be adjusted to an appropriate number by comprehensively considering the specifications of the system, the target execution time of the data processing method, and the like. The user may increase the number of generated light beams to more precisely detect the blank regions B.

Hereinafter, another method of detecting the blank region of the attention region will be described in more detail.

FIG. 8 is a view for explaining another process of detecting the blank region B in the attention region.

Referring to FIG. 8, a sample 3D model M′ may be created through a point cloud, which is a set of a plurality of points P of scan data. The points P may form a polygonal mesh structure. For example, the mesh structure may be a triangular dense structure. At this time, it may be determined that data is not input to a portion where a mesh is not formed between the points P.

More specifically, an outer loop may be formed to externally connect the points P that form the mesh structure in the sample 3D model M′. The outer loop may express the contour of the sample 3D model M′.

Meanwhile, referring to the enlarged view for the X portion in FIG. 8, the sample 3D model M′ may further include closed loop points P′. A region closed by meshes may be created inside the sample 3D model M′ based on the closed loop points P′. That is, the closed loop points P′ may be connected to form an inner loop, and the inner loop may form a closed loop L spaced apart from the outside by meshes. In this case, the inside of the closed loop L is a space for which data is not input, and the inside of the closed loop L (an inner closed loop region) may be detected as a blank region B.

Hereinafter, the low-density region in the attention region will be described in more detail.

The low-density region may refer to an arbitrary region for which scan data less than a critical number of scan data are accumulated. The low-density region may be distinguished into a first low-density region and a second low-density region according to the number of shots of the accumulated scan data. However, the present disclosure is not necessarily limited thereto. In addition, the low-density region may not necessarily be classified into two groups, but may be classified into one or three or more low-density region groups.

Optionally, the low-density region may be calculated based on at least one of the number of scan data and the scan angles between additionally acquired scan data. For example, in the process of obtaining scan data, the location information and scan angle of the scanner may be automatically acquired. At this time, if two or more scan data are collected in real time, a relationship between the respective scan data is derived. In order to derive the relationship between the scan data, a plurality of vertices are extracted from one scan data, a plurality of corresponding points corresponding to the plurality of vertices are calculated from different scan data, a movement function for different scan data is calculated based on one scan data, and data alignment is performed by the change in angle and the translation. At this time, the relative position (location information) and scan angle (angle information) of another scan data may be acquired based on one scan data. Scan data may be accumulated in voxels according to the acquired location and scan angle.

For example, an arbitrary voxel may have first to third angle ranges, and up to 100 pieces of scan data may be accumulated for each angle range. In this case, up to 300 pieces of scan data may be accumulated in one voxel. When 300 pieces of scan data are accumulated, the voxel may be determined to have a density that satisfies the degree of completeness.

That is, even if the same portion of the object is scanned, the reliability of the 3D model may be improved by scanning the corresponding portion at multiple angles. The voxels in which an insufficient amount of scan data is accumulated may be detected as a low-density region.

In the step S140 of determining the degree of completeness, the degree of completeness of the 3D model 200 may be determined based on the ratio of the attention region existing in the entire analysis region 100 described above. For example, in the step S140 of determining the degree of completeness, the 3D model 200 may be determined to be in a complete state when an area or a volume ratio of the attention region to the entire analysis region 100 is less than a predetermined ratio. In this case, the value of the predetermined ratio may be set as a threshold value at which the analysis region 100 has sufficient reliability.

Further, in some cases, in the step S140 of determining the degree of completeness, the degree of completeness may be determined based on the threshold values different for the respective individual tooth regions when the analysis region 100 in the 3D model 200 is distinguished for each individual tooth region. For example, a portion of the analysis region 100 corresponding to the anterior tooth may be determined to be complete only when it has a lower attention region ratio than another portion of the analysis region 100 corresponding to the molar. By applying different threshold values for determining the degree of completeness for each individual tooth region in this way, the overall degree of completeness of the object can be improved, the user’s scan speed can be improved, and the optimal treatment can be provided to the patient.

In addition, according to other cases, in the step S140 of determining the degree of completeness, the degree of completeness may be determined by further subdividing the analysis region 100 in the 3D model 200. For example, in the step S110 of setting the analysis region, a first analysis region having a predetermined threshold value for determining the degree of completeness and a second analysis region having a threshold value smaller than that of the first analysis region may be set. That is, the second analysis region may be set as a region requiring a more precise scan than the first analysis region. The process of setting the second analysis region may be performed according to the user’s selection. For example, a tooth of interest requiring treatment in the individual tooth regions may be set as the second analysis region. Accordingly, the second analysis region may be determined as complete only when it has a lower attention region ratio than the first analysis region, and there is an advantage of inducing a user to more precisely scan a tooth of interest requiring treatment.

Meanwhile, the step S140 of determining the degree of completeness may be performed for the voxels of the 3D model 200 generated in real time, or may be performed by a user clicking an arbitrary button after scanning is completed.

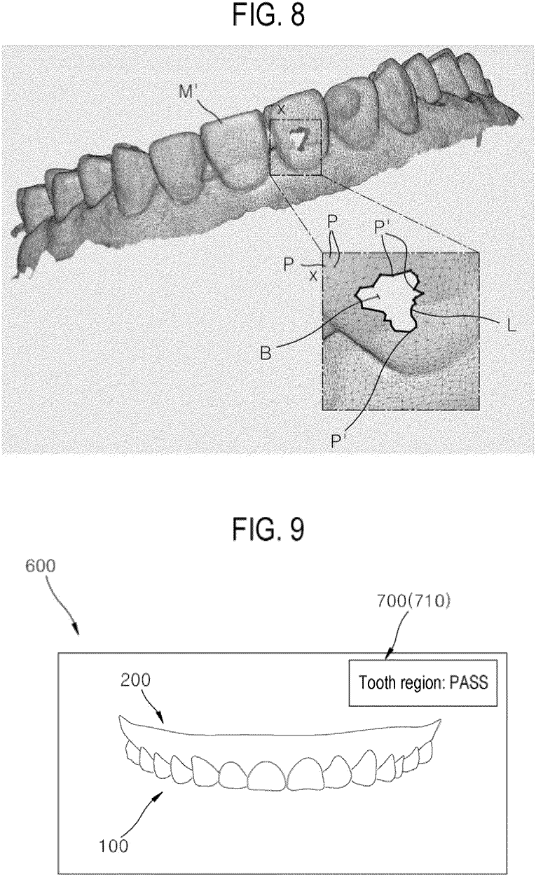

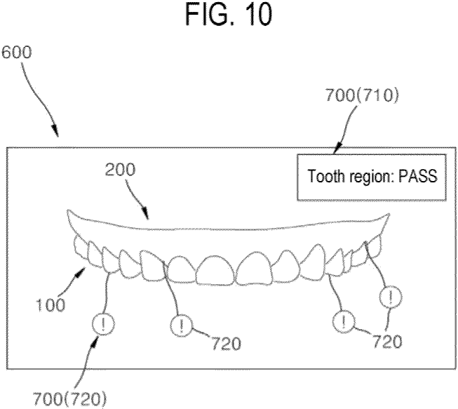

FIG. 9 is a view for explaining the step S150 of generating a feedback based on the result of determining the degree of completeness in the data processing method according to the present disclosure.

Referring to FIG. 9, for example, in the step S150 of generating a feedback, it may be possible to display that the 3D model 200 is completed because the analysis region 100 of the 3D model 200 has sufficient reliability. As shown in FIG. 9, the 3D model 200 and the analysis region 100 applied to the 3D model 200 may be displayed on the user interface screen 600, and a feedback means 700 may be displayed on one side of the user interface screen 600. For example, the feedback means 700 may be a degree of completeness display means 710. The degree of completeness display means 710 may display a message such as “tooth region: PASS” to indicate whether the 3D model 200 is complete. However, the present disclosure is not necessarily limited thereto, and the degree of completeness of the 3D model 200 may be indicated in two or more states (e.g., a complete state, a sufficient state, and an insufficient state). The degree of completeness may be indicated as a numerical value such as percentage (%). A separate loading bar may be displayed on the user interface screen 600 to express the degree of completeness as a graphic change of the loading bar. In addition, the degree of completeness of the 3D model 200 may be displayed in the form of a loading bar and a percentage value. If the degree of completeness exceeds a degree of completeness determination threshold value, a message such as “tooth region: PASS” may be displayed to indicate the completeness of the 3D model 200.

In this way, the user can easily check the completeness of the 3D model 200 through the feedback means 700 even without having to use a conventional reliability map in which the reliability color is indicated on the 3D model 200.

In addition, the feedback means may be a light color and/or a light pattern irradiated by a light projector built in the scanner, in addition to being displayed on the user interface screen 600. For example, when the 3D model 200 is determined to be complete in the step S140 of determining the degree of completeness, the light projector may radiate green light to the surface directed by the scanner. Conversely, when the 3D model 200 is determined not to be complete, the light projector may radiate red light to the surface directed by the scanner. As another example, when the 3D model 200 is determined to be complete in the step S140 of determining the degree of completeness, the light projector may irradiate an “O” pattern to the surface directed by the scanner. Conversely, when the 3D model 200 is determined not to be complete, the light projector may radiate an “X” pattern to the surface directed by the scanner. When the feedback is provided to the user using the light projector built in the scanner in this way, the user can easily understand the completeness of the 3D model 200 without looking at the display device on which the user interface screen 600 is displayed.

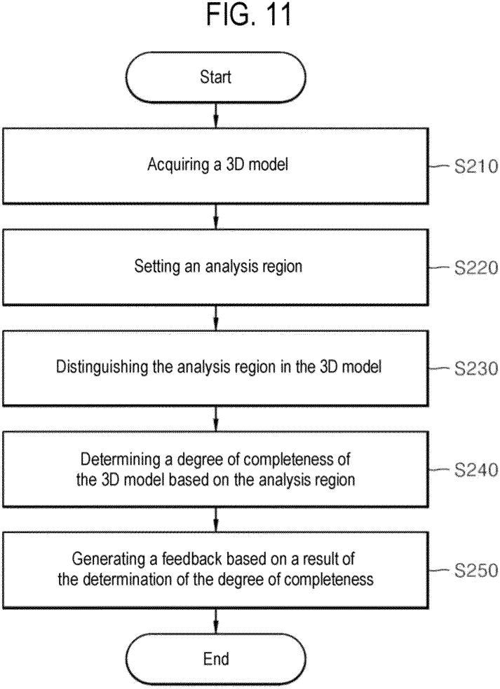

FIG. 10 is a view for explaining the attention region fed back to the user.

Referring to FIG. 10, the user may additionally receive a feedback on the attention region. For example, on the user interface screen 600, the blank region and the low-density region in the analysis region 100 of the 3D model 200 may be visually displayed through an attention region display means 720. The attention region display means 720 may be a predetermined symbol, but is not limited thereto. A virtual scanner shape may be displayed for the attention region. Further, the attention region display means 720 may display the blank region and the low-density region in different forms so as to be distinguished from each other. As the attention region is fed back to the user through the attention region display means 720, the user can easily check a portion of the 3D model 200 that requires additional scanning, and can rapidly improve the degree of completeness of the 3D model 200 by additionally scanning the attention region.

Hereinafter, a data processing method according to another embodiment of the present disclosure will be described. In describing the data processing method according to another embodiment of the present disclosure, the content overlapping with the foregoing content will be briefly mentioned or omitted.

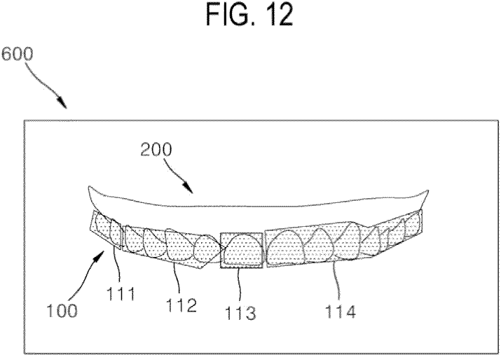

FIG. 11 is a flowchart showing a data processing method according to another embodiment of the present disclosure.

Referring to FIG. 11, the data processing method according to another embodiment of the present disclosure includes a step S210 of acquiring a 3D model. The 3D model may be acquired before setting an analysis region. Accordingly, the shape of the 3D model may be first checked through a user interface screen, and then a portion requiring analysis may be set.

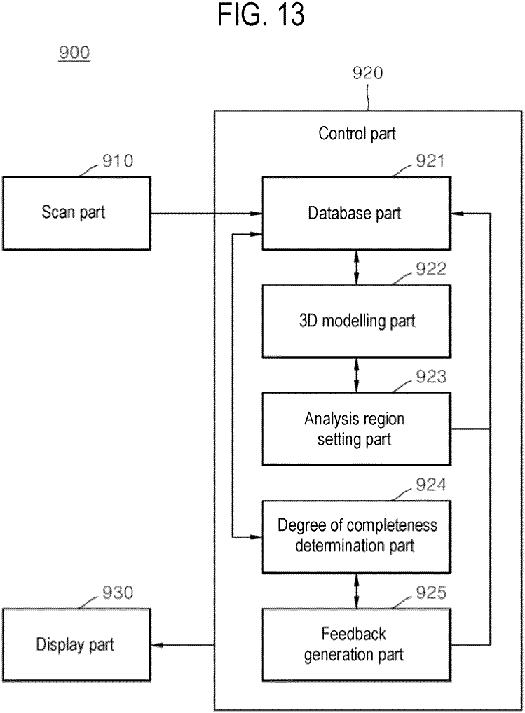

FIG. 12 is a view for explaining a process of setting an analysis region.

Referring to FIGS. 3, 11 and 12, the data processing method according to another embodiment of the present disclosure may include a step S220 of obtaining a 3D model 200 and then setting an analysis region in the obtained 3D model 200. In the step S220 of setting the analysis region, a user may select a tooth to be set as an analysis object on the user interface screen 600 as shown in FIG. 3. Meanwhile, in the data processing method according to another embodiment of the present disclosure, the analysis region may be directly designated on the 3D model because the 3D model has already been obtained. For example, in the data processing method according to another embodiment of the present disclosure, an analysis region may be input on the 3D model, and the analysis region may be set using a brush selection tool or a polygon selection tool. As shown in FIG. 12, the user may select a first analysis region 111, a second analysis region 112, a third analysis region 113, and a fourth analysis region 114 using a polygon selection tool. Meanwhile, the overlapping regions of the analysis regions 111, 112, 113 and 114 may be merged.

Further, predetermined items for setting the analysis region 100 may be displayed. For example, the user may set the analysis region 100 by selecting any one of items displayed on the user interface screen, such as “entire tooth region” and “specific tooth selection.” The process of setting the analysis region 100 is not particularly limited and may use contents other than those listed above. Details of the step of setting the analysis region 100 share the content described above in step S110.

Further, the data processing method according to another embodiment of the present disclosure may include a step S230 of distinguishing the analysis region in the 3D model 200 after the step of setting the analysis region. That is, the data processing method according to another embodiment of the present disclosure may include a step S210 of acquiring a 3D model and a step S220 of setting an analysis region prior to the step S230 of distinguishing an analysis region. In the step S230 of distinguishing an analysis region, the analysis region set by the user using a selection tool provided on the user interface screen may be distinguished, or the analysis region 100 may be distinguished in the 3D model 200 by selecting any one of the predetermined items described above. Details of the step of distinguishing the analysis region share the content described above in step S130.

The data processing method according to another embodiment of the present disclosure may further include a step S240 of determining the degree of completeness of the 3D model 200 based on the distinguished analysis region 100, and a step S250 of generating a feedback based on the result of determining the degree of completeness for the analysis region 100 of the 3D model 200. The step S240 of determining the degree of completeness shares the content described above in step S140, and the step S250 of generating a feedback shares the content described above in step S150.

As can be seen from the above description, by using the data processing method according to the present disclosure, the user can easily acquire a 3D model having sufficient reliability.

In addition, by using the data processing method according to the present disclosure, the degree of completeness is determined for the analysis region 100 rather than the entire 3D model 200. This makes it possible to reduce the data processing time.

In addition, by using the data processing method according to the present disclosure, the user can easily check whether the 3D model 200 has a reliable degree of completeness based on the accurately calculated and determined result without resorting to arbitrary determination.

In addition, by using the data processing method according to the present disclosure, a 3D model 200 can be acquired by precisely scanning a more important tooth by applying different degree of completeness determination thresholds for individual tooth regions.

In addition, by using the data processing method according to the present disclosure, the user can visually and easily check the parts of the 3D model 200 detected as attention regions, and the time and effort required when performing an additional scan to minimize the attention regions can be saved.

In addition, by using the data processing method according to another embodiment of the present disclosure, the advantages of the data processing method according to the present disclosure can be shared and the analysis region 100 can be set directly on the 3D model 200 in detail. This enables the user to precisely set and distinguish only the portion necessary for analysis as the analysis region 100.

Hereinafter, a data processing apparatus that performs the data processing method according to one embodiment of the present disclosure and/or the data processing method according to another embodiment of the present disclosure will be described. In describing the data processing apparatus according to the present disclosure, overlapping contents are briefly described or omitted.

FIG. 13 is a schematic configuration diagram showing a data processing apparatus 900 that performs the data processing method according to the present disclosure.

Referring to FIG. 13, the data processing apparatus 900 according to the present invention includes a scan part 910, a control part 920, and a display part 930.

The scan part 910 may acquire an image of an object by scanning the object. The object may be a tooth of a patient. The scan part 910 may perform at least a part of the step of obtaining a 3D model in the above-described data processing method. The scan part 910 may be the above-described scanner (e.g., a 3D scanner).

Hereinafter, a detailed configuration of the control part 920 will be described.

The control part 920 may create a 3D model based on the image of the object obtained from the scan part 910, may determine a degree of completeness of the 3D model, and may provide a feedback to the user. For example, the control part 920 may include a microprocessor for data operation processing.

The control part 920 may include a database part 921. The image and characteristic information (reliability, scan angle, location, etc.) of the object acquired by the scan part 910, the 3D model of the object created from a 3D modeling part 922 described later, the standard oral cavity shape used when setting an analysis region, the alignment algorithm for overlapping and aligning scan data, the 3D model degree of completeness determination algorithm, the criterion for determining an attention region, the criterion for generating a feedback, and the like may be stored in the database part 921. The database part 921 may be a known data storage means, and may be at least one of known storage means including a Solid State Drive (SSD) and a Hard Disk Drive (HDD). In addition, the database part 921 may be a virtual cloud storage means.

The control part 920 may include a 3D modeling part 922. The 3D modeling part 922 may model 2D images of the object obtained from the scan part 910 into a 3D model. Meanwhile, the 3D modeling part 922 may align the 3D-modeled scan data (more specifically, 3D data shots) according to the alignment algorithm stored in the database part 921. A 3D model representing the object may be generated through alignment and merging.

In addition, the control part 920 may further include an analysis region setting part 923. The analysis region setting part 923 may set an analysis region for determining the degree of completeness in the 3D model. At this time, the analysis region setting part 923 may set an analysis region in the standard oral cavity shape before the 3D model is acquired, and may apply the analysis region to a 3D model acquired later. Alternatively, the 3D model may be acquired and then the analysis region may be applied directly to the 3D model.

In addition, the control part 920 may include a degree of completeness determination part 924. The degree of completeness determination part 924 may determine that the 3D model is complete when the ratio of the attention region to the entire analysis region in the analysis region of the acquired 3D model is equal to or less than a predetermined threshold value. The attention region may include a blank region and a low-density region. Meanwhile, the degree of completeness determination part 924 may apply different degree of completeness determination threshold values to individual tooth regions. The related descriptions are the same as described above.

In addition, the control part 920 may further include a feedback generation part 925. The feedback generation part 925 may provide a feedback of the degree of completeness determination result to the user based on the degree of completeness determination result of the degree of completeness determination part 924. At this time, the degree of completeness determination result may be expressed in at least one of various forms such as a degree of completeness display means including a message such as “Tooth region: PASS”, a degree of completeness percentage, and a loading bar.

The feedback generation part 925 may display the attention region on the 3D model. The feedback generation part 925 may provide a feedback of the attention region to the user through a predetermined symbol or a virtual scanner shape. In this way, the feedback generation part 925 allows the user to easily check the attention region. The user can additionally scan the attention region to quickly improve the degree of completeness of the 3D model.

Meanwhile, the data processing apparatus 900 according to the present disclosure may include a display part 930. The display part 930 may visually display an image of an object acquired by the scan part 910 and at least a part of a series of processes performed by the control part 920. A known device may be used as the display part 930 to display a data processing process to the user. For example, the display part 930 may be one of visual display devices such as a monitor and a tablet. The user can check the data processing process displayed on the display part 930 and can easily obtain various information such as whether the 3D model has been acquired to have a sufficient degree of completeness in the analysis region and where the location of the attention region is.

As described above, by using the data processing apparatus that performs the data processing method according to one embodiment of the present disclosure and the data processing method according to another embodiment of the present disclosure, the user can enjoy the advantages obtained by performing the above-described data processing method.

The above description is merely an exemplary description of the technical idea of the present disclosure, and various modifications and variations may be made by those skilled in the art without departing from the essential features of the present disclosure.

Therefore, the embodiments disclosed herein are not intended to limit the technical idea of the present disclosure, but to explain the technical idea of the present disclosure. The scope of the technical idea of the present disclosure is not limited by these embodiments. The protection scope of the present disclosure should be construed according to the appended claims, and all technical ideas falling within the equivalent range should be construed as being included in the scope of the present disclosure.

The present disclosure provides a data processing method that allows the user to intuitively check the degree of completeness of an analysis region by setting an analysis region of a 3D model.

What is claimed is:

1. A data processing method, comprising:
distinguishing, in a 3D model, an analysis region including at least one tooth region; and
determining a degree of completeness of the 3D model based on the analysis region.

2. The method of claim 1, further comprising:
before distinguishing the analysis region, setting the analysis region; and
acquiring the 3D model after setting the analysis region.

3. The method of claim 1, further comprising:
before distinguishing the analysis region, acquiring the 3D model; and
setting the analysis region in the acquired 3D model.

4. The method of claim 1, wherein the tooth region includes an entire tooth region of the 3D model.

5. The method of claim 1, wherein the 3D model is distinguished into the tooth region representing teeth and a gingival region representing gingivae through at least one of color information and curvature information.

6. The method of claim 1, wherein in distinguishing the analysis region, the tooth region is distinguished into individual tooth regions representing individual teeth according to a dental formula distinguishing criterion including surface curvature information of a tooth.

7. The method of claim 6, wherein the dental formula distinguishing criterion further includes at least one of size information and shape information of the tooth.

8. The method of claim 1, wherein in determining the degree of completeness, the 3D model is determined to be complete when an area or a volume ratio of an attention region to the analysis region is less than a predetermined ratio.

9. The method of claim 8, wherein the attention region includes at least one of a blank region in the 3D model for which scan data is not input and a low-density region in the 3D model for which scan data is input as being below a predetermined threshold density.

10. The method of claim 8, wherein in determining the degree of completeness, the degree of completeness is determined based on a threshold value different for each individual tooth region of the 3D model.

11. The method of claim 9, wherein the blank region includes a hole region detected by aligning the 3D model with a pre-stored template and using an intersection test through at least one light beam generated from the surface of the template.

12. The method of claim 9, wherein the blank region includes an inner closed-loop region created by boundaries of scan data constituting the 3D model.

13. The method of claim 9, wherein the low-density region is calculated based on at least one of a number of acquired scan data and a scan angle between the scan data.

14. The method of claim 1, further comprising:
generating a feedback to a user based on the result of determining the degree of completeness of the 3D model.

15. The method of claim 8, wherein the attention region is fed back to a user.
